(19) United States
(12) Patent Application Publication
(10) Pub. No.: US 2019/0289805 A1
(43) Pub. Date: Sep. 26, 2019

(54) CULTURING METHOD FOR CULTURED LEGUMINOUS ROOTS HAVING INCREASED COUMESTROL CONTENT

(71) Applicants: Amorepacific Corporation, Seoul (KR); Chungbuk National University Industry-Academic Co Foundation, Cheongju-si, Chungcheongbuk-do (KR)

(72) Inventors: Eun Jung LEE, Yongin-si, Gyeonggi-do (KR); Young-Gyu KANG, Yongin-si, Gyeonggi-do (KR); Jun Seong PARK, Yongin-si, Gyeonggi-do (KR); So Young PARK, Cheongju-si, Chungscheongbuk-do (KR); Young Eun KIM, Sejong (KR)

(21) Appl. No.: 16/317,414

(22) PCT Filed: Jul. 10, 2017
(86) PCT No.: PCT/KR2017/007356

§ 371 Date: Jan. 11, 2019

(30) Foreign Application Priority Data
Jul. 14, 2016 (KR) 10-2016-0089464
Jun. 29, 2017 (KR) 10-2017-0082586

(51) Int. Cl. A01H 4/00; A01H 5/10; C12N 5/04; C12N 5/00

(52) U.S. Cl. CPC A01H 4/005; C12N 5/0025; C12N 5/04; A01H 5/10

(57) ABSTRACT

The present specification relates to a culturing method for cultured leguminous roots having an increased coumestrol content, the method being capable of mass-producing coumestrol, which is present in a very small amount in a leguminous plant, wherein the culturing method comprises the steps of: (a) germinating leguminous seeds in a culture medium to induce in vitro plants having cotyledons, hypocotyls, and radicles; (b) culturing, in a culture medium, at least one site of cotyledons, hypocotyls, and radicles of the induced in vitro plant to induce site-specific cultured roots; and (c) multiplying the induced site-specific cultured roots in a culture medium, wherein the culture medium contains nutrient components of NH₄NO₃, CaCl₂.2H₂O, MgSO₄.7H₂O, KH₂PO₄, and KNO₃.

### TECHNICAL FIELD

The present disclosure relates to a culturing method for cultured leguminous roots having increased coumestrol content, and cultured leguminous roots having increased coumestrol content obtained by said method.

### BACKGROUND ART

To date, coumestrol has been known as the strongest substance among other vegetable estrogens. It is mainly found in seeds, roots, and leaves of leguminosae and compositae plants, and is generally classified as coumestan series compound and is a type of isoflavonoid. The coumestrol has come to attention due to the fact that it is secreted at a high concentration in an injured site when a plant is damaged, and has anti-fungal and anti-viral activities through anti-oxidative, anti-inflammatory, and anti-toxic activities to prevent infection. The reason for this is that infection due to various bacteria, fungus and viruses induces the synthesis of various aromatic compounds including coumestrol. Such antibiotic activity of coumestrol is based on its phenolic structure, which is a chemical framework for an antioxidant, and thus the introduction of free radical oxidants is inhibited and the production of peroxide in body is blocked. Furthermore, only coumestrol is known to have an estrogen effect among many other natural coumestan derivatives. An estrogen effect was evaluated based on a change in weight of uterus after the oral administration of coumestrol to an immature mouse. From the test results, it was observed that coumestrol exhibits an effective estrogen effect in young female mice, but has no estrogen effect in mature male mice. Also, it was demonstrated that coumestrol has no toxicity.

Currently, commercial natural coumestrol is highly expensive since coumestrol is present in a very small amount in leguminosae plants. For this reason, there were attempts to obtain coumestrol through synthesis. However, an approach to develop a simple synthesis method for coumestrol is yet to be found due to a complicated chemical structure wherein a number of aromatic rings are fused. Although several synthesis methods have been reported, each method has a variety of problems and thus makes commercialization challenging. Moreover, natural components are considered to be better in safety than chemically synthesized components, so a method for mass-production from natural components is being developed.

Therefore, the inventors have studied a method for naturally mass-producing coumestrol and have completed the present invention.

### SUMMARY OF INVENTION

### Technical Problem

To solve the aforementioned issues, the present inventors have completed the present invention relating to a culturing method for cultured leguminous roots having increased coumestrol content and cultured leguminous roots having increased coumestrol content.

According to an aspect of the present invention, there is provided a culturing method for cultured leguminous roots having increased coumestrol content.

According to an aspect of the present invention, there is provided a culturing method for cultured leguminous roots having increased coumestrol content which is capable of producing a constant amount of coumestrol throughout the year.

According to an aspect of the present invention, there is provided a culturing method for cultured leguminous roots having increased coumestrol content which is capable of mass-producing coumestrol.

According to another aspect of the present invention, there is provided cultured leguminous roots having increased coumestrol content.

### Solution to Problem

According to an aspect, the present invention provides a culturing method for cultured leguminous roots having increased coumestrol content comprising the steps of:

(a) germinating leguminous seeds in a culture medium to induce in vitro plants having cotyledons, hypocotyls, and radicles;

(b) culturing, in a culture medium, at least one site of cotyledons, hypocotyls, and radicles of the induced in vitro plant to induce site-specific cultured roots; and

(c) multiplying the induced site-specific cultured roots in a culture medium, wherein the culture medium contains nutrient components of NH₄NO₃, CaCl₂.2H₂O, MgSO₄.7H₂O, KH₂PO₄, and KNO₃.

According to an aspect, the concentration of NH₄NO₃ in the culture medium may be between 1,500 and 2,000 mg/L, the concentration of CaCl₂.2H₂O in the culture medium may be between 300 and 500 mg/L, the concentration of MgSO₄.7H₂O in the culture medium may be between 300 and 500 mg/L, the concentration of KH₂PO₄ in the culture medium may be between 100 and 200 mg/L, and the concentration of KNO₃ in the culture medium may be between 1,700 and 2,100 mg/L.

According to an aspect, the culture medium may be a MS medium (Murashige and Skoog medium).

According to an aspect, in the step (a), the leguminous seeds are from at least one of Phynchosia nulubilis Loureiro, Glycine max Merr., Glycine Max Merr., and Glycine gracillis.

According to an aspect, in the step (a), the culture medium contains 10-100 g/L of sucrose based on a total volume of the medium.

According to an aspect, in the step (b), the culture medium contains at least one of IBA (indole butyric acid) and NAA (naphthalene acetic acid) at an amount between 0.1 to 10 mg/L based on the total volume of the medium, and the culture medium contains 10-100 g/L of sucrose based on the total volume of the medium.

According to an aspect, in the step (b), the culture medium contains 2 to 8 mg/L of IBA (indole butyric acid) based on the total volume of the medium.

According to an aspect, the leguminous seeds in the step (a) are from at least one of Glycine Max Merr and Glycine gracillis; the induced site in the step (b) is at least one of hypocotyls and radicles; and the culture medium in the step (b) contains 3 to 5 mg/L of IBA (indole butyric acid) based on the total volume of the medium.

According to an aspect, in the step (c), the culture medium contains 3 to 5 mg/L of IBA (indole butyric acid) and 10-100 g/L of sucrose based on the total volume of the medium.

According to an aspect, in the step (c), the culture medium is 0.5-1.5 MS medium and contains 30-60 g/L of sucrose.

According to another aspect, the present invention provides cultured leguminous roots having increased coumestrol content, produced by any one of the aforementioned methods.

### Advantageous Effects of Invention

In an aspect, the culturing method according to the present invention is capable of mass-producing coumestrol.

In an aspect, the culturing method according to the present invention is capable of producing a constant amount of coumestrol throughout the year.

In an aspect, the culturing method according to the present invention is capable of producing a large amount of coumestrol in a short time.

In another aspect, the cultured leguminous roots according to the present invention have increased coumestrol content.

In another aspect, the cultured leguminous roots according to the present invention have uniform coumestrol content.

### BRIEF DESCRIPTION OF DRAWINGS

FIG. 1 is a schematic view illustrating the germination of leguminous seeds and the induction of in vitro plant in accordance with Preparation Example 1-1.

FIGS. 2 and 3 show the induction status after 2 weeks of culturing depending on the type of bean, the induced site of in vitro plant, and the type and amount of auxin, in accordance with Preparation Example 1-2.

FIGS. 4 and 5 show the growth status of the cultured leguminous roots depending on the type of bean and the induced site, and the result of measuring the content of coumestrol in cultured leguminous roots, in accordance with Experimental Example 1.

FIG. 6 shows the result of an optimum medium composition for coumestrol multiplication in the step of multiplying cultured leguminous roots in the Preparation Example 1-3, as measured by a DOE method.

### DESCRIPTION OF EMBODIMENTS

Hereinafter, the present invention will be described in detail.

The coumestrol is a primary bioactive substance of soybean. It is present in various amounts in leguminous plant depending on its external environment as well as in largely differential amount depending on genus/species/variety thereof. Especially, it is present in a very small amount in a plant. When coumestrol is produced using cultured leguminous roots induced by the culturing method according to the present invention, coumestrol can be produced at a constant amount throughout the year. Also, the mass-production of in vitro coumestrol can be achieved by a control of culture process.

According to an aspect, the present invention provides a culturing method for cultured leguminous roots having increased coumestrol content comprising the steps of:

(a) germinating leguminous seeds in a culture medium to induce in vitro plants having cotyledons, hypocotyls, and radicles;

(b) culturing, in a culture medium, at least one site of cotyledons, hypocotyls, and radicles of the induced in vitro plant to induce site-specific cultured roots; and

(c) multiplying the induced site-specific cultured roots in a culture medium,

wherein the culture medium contains nutrient components of NH₄NO₃, CaCl₂.2H₂O, MgSO₄.7H₂O, KH₂PO₄, and KNO₃.

As used herein, “a leguminous plant” may be any leguminous plant which is capable of producing coumestrol.

As used herein, seeds of leguminous plant are seeds from any leguminous plant. In particular, seeds of leguminous plant may be “bean”.

According to an aspect, in the step (a), the leguminous plant may be at least one of Phynchosia nulubilis Loureiro, Glycine max Merr., Glycine Max Merr., and Glycine gracillis.

In this specification, “a culturing method for cultured leguminous roots” refers to a method of extracting at least one of a specific cell, tissue and organ from any leguminous plant and culturing in vitro in a culture medium containing nutrients under an aseptic condition to regenerate callus or single cells as an organic or complete plant.

Such culturing method for cultured plant or leguminous roots may be called plant or leguminous plant explanation, tissue culture, in vitro culture, aseptic culture, or plant stem-cell culture.

According to an aspect, the concentration of NH₄NO₃ in the culture medium may be between 1,500 and 2,000 mg/L, the concentration of CaCl₂.2H₂O in the culture medium may be between 300 and 500 mg/L, the concentration of MgSO₄.7H₂O in the culture medium may be between 300 and 500 mg/L, the concentration of KH₂PO₄ in the culture medium may be between 100 and 200 mg/L, and the concentration of KNO₃ in the culture medium may be between 1,700 and 2,100 mg/L.

According to an aspect, the culture medium may be a MS medium (Murashige and Skoog medium).

According to an aspect, the culture medium may be a MS medium (Murashige and Skoog medium). Specifically, according to a concentration of inorganic substance in the medium, ¼ MS, ½ MS, ¾ MS, 1 MS, 3/2 MS or 2 MS medium may be used.

According to an aspect, in the step (a), the culture medium contains 10-100 g/L of sucrose based on the total volume of the medium.

Specifically, the concentration of sucrose may be at least 10 g/L, at least 20 g/L, at least 21 g/L, at least 22 g/L, at least 23 g/L, at least 24 g/L, at least 25 g/L, at least 26 g/L, at least 27 g/L, at least 28 g/L, at least 29 g/L, or at least 30 g/L based on the total volume of the culture medium. Further, the concentration of sucrose may be 100 g/L or less, 80 g/L or less, 60 g/L or less, 50 g/L or less, 40 g/L or less, 39 g/L or less, 38 g/L or less, 37 g/L or less, 36 g/L or less, 35 g/L or less, 34 g/L or less, 33 g/L or less, 32 g/L or less, 31 g/L or less, or 30 g/L or less based on the total volume of the culture medium. When the concentration of sucrose in the step (a) is within the range indicated above, a high induction of in vitro plant can be achieved.

According to an aspect, the induction of in vitro plant in the step (a) may be carried out until cotyledons, hypocotyls, and radicles are generated from seeds. Such induction of in vitro plant in the step (a) may be carried out for 1 to 25 days. Specifically, the induction of in vitro plant in the step (a) may be carried out for at least 1 day, at least 1.5 days, at least 2 days, at least 2.5 days, at least 7 days, at least 8 days, at least 9 days, at least 10 days, at least 11 days, at least 12 days, at least 13 days, at least 14 days, or at least 15 days. Further, the induction may be carried out for 25 days or less, 24 days or less, 23 days or less, 22 days or less, 21 days or less, 20 days or less, 19 days or less, 18 days or less, 17 days or less or 16 days or less.

According to an aspect, the induction of in vitro plant in the step (a) may be carried out until the length of in vitro plant becomes 8 to 15 cm. As used herein, “length of in vitro plant” refers to a straight length from an end of cotyledon to an end of radical.

Specifically, the length may be at least 8 cm, at least 9 cm, at least 10 cm, or at least 11 cm, and also may be 15 cm or less, 14 cm or less or 13 cm or less.

According to an aspect, the induction of in vitro plant in the step (a) may be carried out under light condition.

According to an aspect, in the step (b), the culture medium contains at least one of IBA (indole butyric acid) and NAA (naphthalene acetic acid) at an amount between 0.1 to 10 mg/L based on the total volume of the culture medium, and the culture medium contains 10-100 g/L of sucrose based on the total volume of the medium.

The concentration of IBA or NAA may be at least 0.1 mg/L, at least 0.5 mg/L, at least 1 mg/L, at least 2 mg/L, at least 3 mg/L, at least 3.5 mg/L, at least 3.6 mg/L, at least 3.7 mg/L, at least 3.8 mg/L, at least 3.9 mg/L, at least 4 mg/L, or at least 5 mg/L. Further, the concentration of IBA or NAA may be 10 mg/L or less, 9 mg/L or less, 8 mg/L or less, 7 mg/L or less, 6 mg/L or less, 5 mg/L or less, 4.5 mg/L or less, 4.3 mg/L or less, 4.1 mg/L or less, 3.5 mg/L or less or 3 mg/L or less.

According to an aspect, in the step (b), the culture medium contains 2 to 8 mg/L of IBA (indole butyric acid) based on the total volume of the culture medium. Specifically, IBA (indole butyric acid) may be contained at an amount of at least 2 mg/L, at least 3 mg/L, at least 3.6 mg/L, at least 3.7 mg/L, at least 3.8 mg/L, at least 3.9 mg/L, at least 4 mg/L, or at least 5 mg/L, and also at an amount of 8 mg/L or less, 7 mg/L or less, 6 mg/L or less, 5 mg/L or less, 4.5 mg/L or less, 4.3 mg/L or less, 4.1 mg/L or less, 3.5 mg/L or less, or 3 mg/L or less based on the total volume of the culture medium.

According to an aspect, the leguminous seeds in the step (a) are from at least one of Glycine Max Merr and Glycine gracillis; the induced site in the step (b) is at least one of hypocotyls and radicles; and the culture medium in the step (b) contains 3 to 5 mg/L of IBA (indole butyric acid) based on the total volume of the culture medium.

According to an aspect, in the step (c), the culture medium contains 3 to 5 mg/L of IBA (indole butyric acid) and 10-100 g/L of sucrose based on the total volume of the culture medium.

According to an aspect, in the step (c), the culture medium is 0.5-1.5 MS medium and contains 30-60 g/L of sucrose.

Specifically, the concentration of sucrose in the step (c) may be at least 10 g/L, at least 20 g/L, at least 21 g/L, at least 22 g/L, at least 23 g/L, at least 24 g/L, at least 25 g/L, at least 26 g/L, at least 27 g/L, at least 28 g/L, at least 29 g/L, or at least 30 g/L based on the total volume of the culture medium. Further, the concentration of sucrose may be 100 g/L or less, 80 g/L or less, 60 g/L or less, 50 g/L or less, 40 g/L or less, 39 g/L or less, 38 g/L or less, 37 g/L or less, 36 g/L or less, 35 g/L or less, 34 g/L or less, 33 g/L or less, 32 g/L or less, 31 g/L, or less or 30 g/L or less based on the total volume of the culture medium. When the concentration of sucrose in the step (c) is within the range indicated above, a high multiplication of cultured roots can be achieved, and the content of coumestrol in cultured roots can be increased.

Moreover, the culture medium in the step (c) may be 0.5 MS, 0.6 MS, 0.7 MS, 0.75 MS, 0.8 MS, 0.9 MS, 1.0 MS, or 1.5 MS medium according to the concentration of inorganic substance in the medium. When MS medium according to the concentration of inorganic substance in the step (c) is at least one of MS media listed above, a high multiplication of cultured roots can be achieved, and the content of coumestrol in cultured roots can be increased.

According to another aspect, the present invention provides cultured leguminous roots having increased coumestrol content, produced by any one of the aforementioned methods.

According to another aspect, the content of coumestrol in the cultured roots is at least 0.001 wt % with respect to the total dry weight of the cultured roots. Specifically, the content of coumestrol in the cultured roots may be at least 0.001 wt %, at least 0.002 wt %, at least 0.003 wt %, at least 0.004 wt %, at least 0.005 wt %, at least 0.006 wt %, at least 0.007 wt %, at least 0.008 wt %, at least 0.009 wt %, at least 0.01 wt %, at least 0.012 wt %, at least 0.014 wt %, at least 0.015 wt %, at least 0.016 wt %, or at least 0.017 wt % with respect to the total dry weight of the cultured roots. Further, the content of coumestrol in the cultured roots may be 1 wt % or less, 0.9 wt % or less, 0.8 wt % or less, 0.6 wt % or less, 0.5 wt % or less, 0.4 wt % or less, 0.3 wt % or less, or 0.2 wt % or less with respect to the total dry weight of the cultured roots. In an embodiment, cultured roots cultured according to the culturing method for cultured leguminous roots according to the present invention may contain 0.01-0.02 wt % of coumestrol with respect to the total dry weight of the cultured roots.

### EXAMPLES

Hereinafter, the present invention will be described in detail with reference to Examples. It will be obvious to those skilled in the art that these examples are only for illustrating the present invention but are not intended to limit the scope of the invention.

### [Preparation Example 1-1] Germination of Bean Seeds and In Vitro Induction of Plant

Each surface of Glycine Max Merr. and Glycine gracillis seeds was sterilized using 2% sodium hypochlorite for 20 minutes and washed 3 times with sterile water. Then, a plant was induced in ½ MS medium (Murashige and Skoog Medium) supplemented with 30 g/L of sucrose under light condition at 25±1° C. for 2-3 weeks.

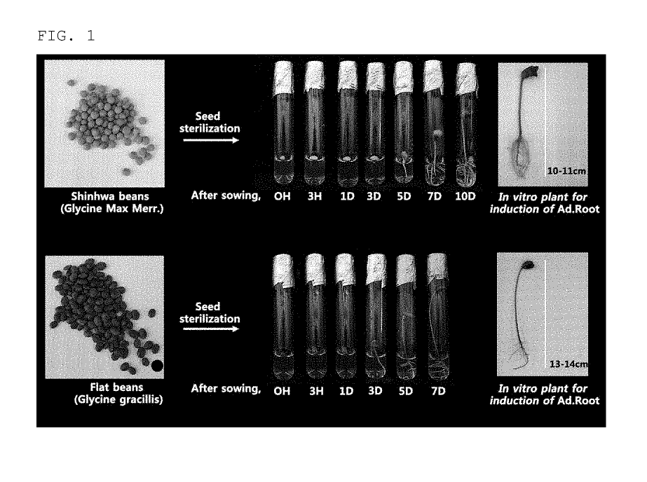

The procedure of inducing an in vitro plant from leguminous seeds is shown in FIG. 1.

### [Preparation Example 1-2] Induction of Cultured Bean Roots

Cotyledons, hypocotyls, and radicles from the induced plant were cut to about 1 cm and maintained in 1 MS medium supplemented with 30 g/L of sucrose with 0, 1.0, 2.0, 4.0, 8.0 mg/L of IBA (indole butyric acid) and NAA (naphthalene acetic acid), respectively, under dark condition at 22±1° C. for 2-3 weeks to induce cultured roots.

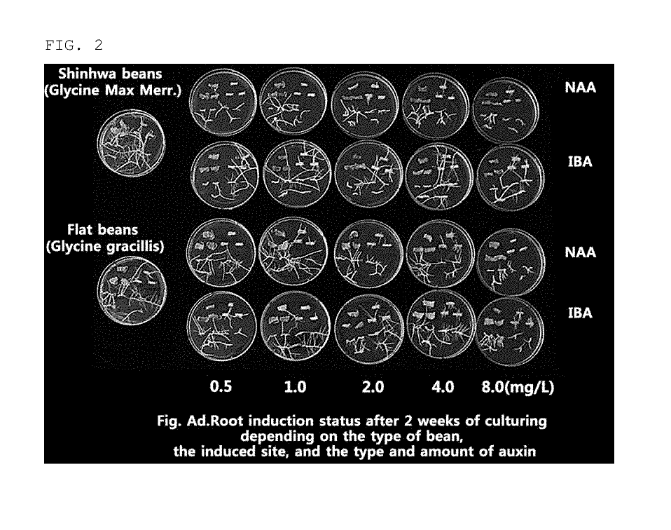

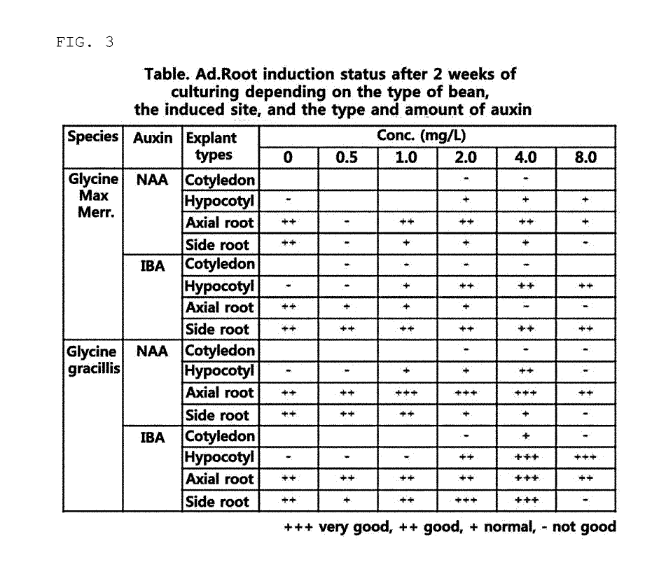

The induction status depending on the type of bean, the induced site, and the type and amount of auxin are shown in FIGS. 2 and 3.

### [Experimental Example 1] Measurement of Dry Substance Productivity and Coumestrol Content of Cultured Roots Depending on the Variety of Bean, the Induced Site, and the Type and Amount of Auxin

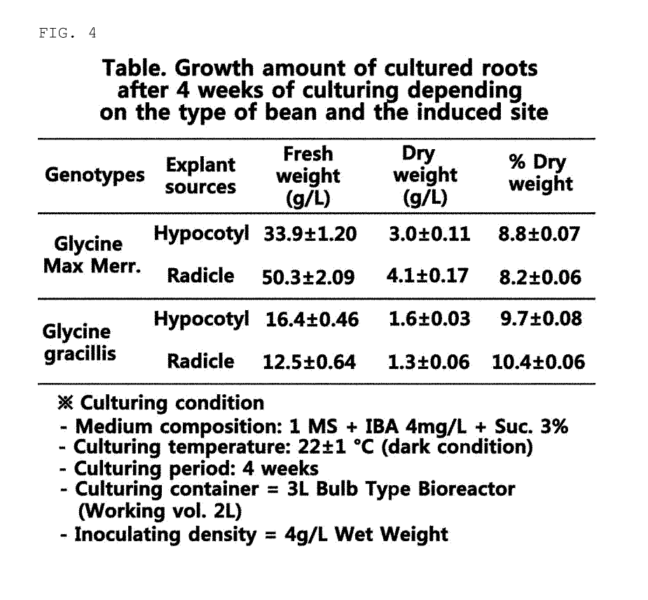

Dry substance productivity from dry cultured bean roots of the Preparation Example 1-2 was measured. The result is shown in FIG. 4. Growth statuses of cultured bean roots were different depending on the type of bean and the induced site. In particular, the net weight of cultured roots derived from radicles of Glycine Max Merr. was increased by 12.5 times with respect to the initial value after 4 weeks of culture. The final dry substance productivity was also excellent as 4.1 g/L.

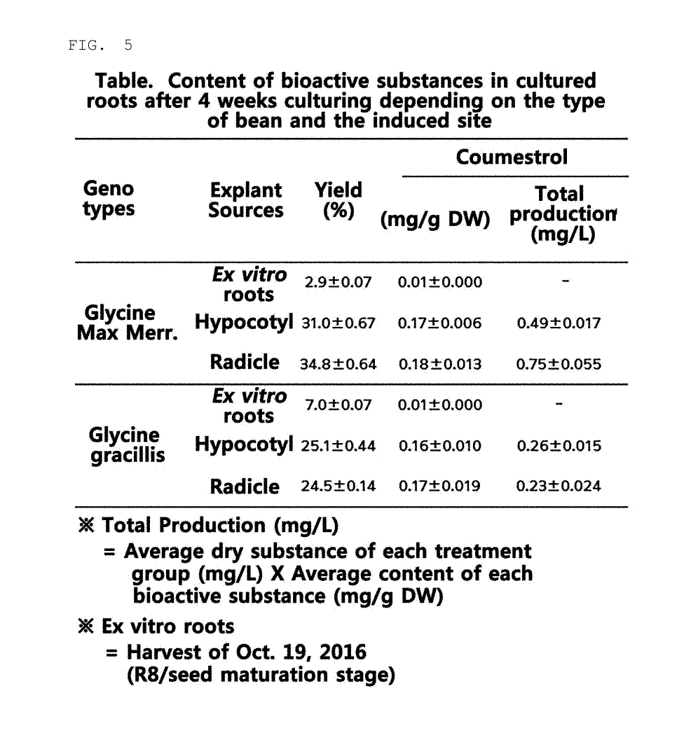

Further, the dry cultured bean roots of Preparation Example 1-2 were extracted in 80% (w/v) ethanol at room temperature for 24 hours. The resulting extract was filtered using a filter paper and dried by evaporating a solvent to obtain a powder. Then, the powder was diluted to be a 1% solution to prepare a final extract. To measure the content of coumestrol in the cultured roots, 2 mL of the extract was filtered with 0.45 μm filter and injected into a high-performance liquid chromatography with a UV detector by 10 μL. A column of Mightysil RP-18 GP 250-4.6 (5 μm) column was used. The content of coumestrol in the extract was measured at 342 nm wavelength. The result is shown in FIG. 5. As seen in FIG. 5, the coumestrol content was the highest as 0.18 mg/g DW in the cultured roots derived from radicles of Glycine Max Merr.

Based on the experimental results indicated above, the cultured roots derived from radicles of Glycine Max Merr. having the highest coumestrol content were multiplied using the method in Preparation Example 1-3 as described below.

### [Preparation Example 1-3] Optimum Medium Condition for Multiplication of Cultured Bean Roots Using DOE Method and Multiplication Using Said Medium

When carrying out multiplication in a bioreactor, the medium composition suitable for coumestrol production may be varied depending on the medium condition (concentrations of inorganic substance and sucrose). Thus, using cultured roots derived from radicles of Glycine Max Merr. which is determined to be most suitable for producing coumestrol, an optimum medium composition was selected by DOE method, and the cultured roots were multiplied in the selected medium.

(1) Selection of Optimum Medium Composition for Multiplication of Cultured Roots

By using Design of Experiments (DOE) method, an experiment for optimizing a medium composition was performed. The condition of the experiment was as follows:

2 factors×3 levels×2 repetitions (treatment factor:sucrose×MS Salt/treatment level:sucrose=1−9%/MS Salt=0.25−2 times)

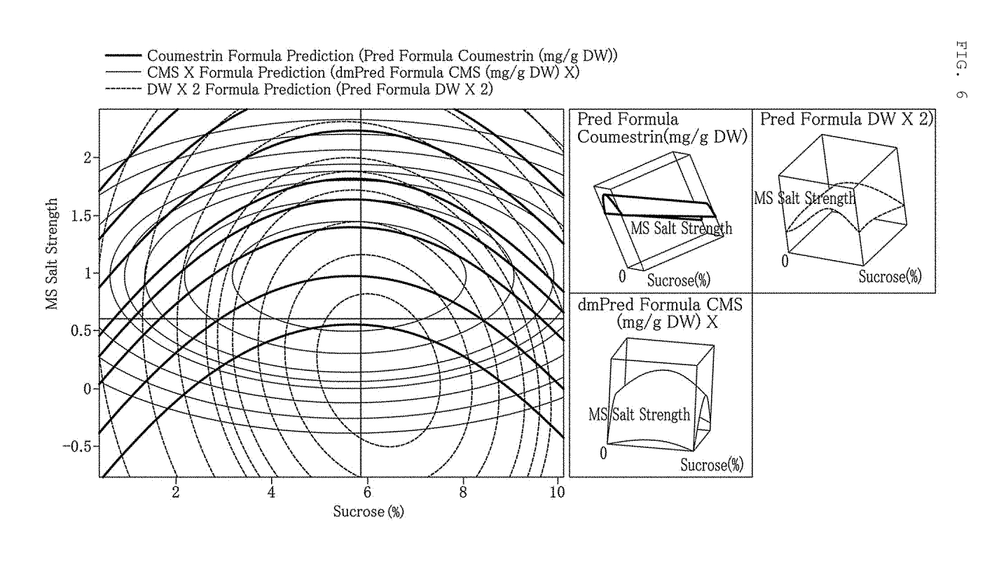

The result from the prediction models obtained according to said method is shown in FIG. 6. From this result, it was demonstrated that the optimum medium composition has 0.5-1 MS of inorganic substance and 3-6% of sucrose in the medium.

(2) Multiplication of Cultured Roots in the Optimum Medium

The cultured roots of Preparation Example 1-2 were multiplied at 3 to 4-week intervals using 2 L of 1 MS medium with 4 mg/L of IBA and 30 g/L of sucrose in a bulb type bioreactor with 3 L air volume. The medium was used after adjusting the pH to 5.8 using 1N NaOH and sterilizing at 121° C. and 1.2 atmospheric pressure for 35 minutes. The cultured roots were cultured by cutting them to 1 to 1.5 cm, inoculating the cultured roots at an inoculation density of 4-5 g/L with respect to its net weight, and culturing under dark condition at 22±1° C. Air was supplied at a constant amount of 0.1 vvm throughout the culture process. To maintain the temperature temperature of air provided in the bioreactor constant, air was sequentially passed through an air compressor capable of condensing compressed air, a filter capable of removing impurities, and an air drier, and then supplied to the bioreactor using an oil-free air compressor.

### [Experimental Example 2] Measurement of Coumestrol Content in Multiplied Bean Roots

The content of coumestrol was measured on bean roots multiplied in Preparation Example 1-3 using the same method as in Experimental Example 1.

The coumestrol content was measured to be 0.16 mg/g dry weight (0.016 wt %) with respect to the dry weight of the multiplied bean roots.

While the present invention has been described with reference to specific embodiments thereof, but it is to be understood by a person skilled in the art that these specific embodiments are merely preferred embodiments and that the scope of the present invention is not limited thereby. Accordingly, the scope of the present invention will be defined by the appended claims and their equivalents.

1. A culturing method for cultured leguminous roots having increased coumestrol content comprising the steps of:
(a) germinating leguminous seeds in a culture medium to induce in vitro plants having cotyledons, hypocotyls, and radicles;
(b) culturing, in a culture medium, at least one site of cotyledons, hypocotyls, and radicles of the induced in vitro plant to induce site-specific cultured roots; and
(c) multiplying the induced site-specific cultured roots in a culture medium,
wherein the culture medium contains nutrient components of NH₄NO₃, CaCl₂.2H₂O, MgSO₄.7H₂O, KH₂PO₄, and KNO₃.

2. The culturing method of claim 1, wherein the concentration of NH₄NO₃ in the culture medium is between 1,500 and 2,000 mg/L, the concentration of CaCl₂.2H₂O in the culture medium is between 300 and 500 mg/L, the concentration of MgSO₄.7H₂O in the culture medium is between 300 and 500 mg/L, the concentration of KH₂PO₄ in the culture medium is between 100 and 200 mg/L, and the concentration of KNO₃ in the culture medium is between 1,700 and 2,100 mg/L.

3. The culturing method of claim 2, wherein the culture medium is MS medium (Murashige and Skoog medium).

4. The culturing method of claim 1, wherein in the step (a), the leguminous seeds are from at least one of Phynchosia nulubilis Loureiro, Glycine max Merr., Glycine Max Merr., and Glycine gracillis.

5. The culturing method of claim 1, wherein in the step (a), the culture medium contains 10-100 g/L of sucrose based on the total volume of the culture medium.

6. The culturing method of claim 1, wherein in the step (b), the culture medium contains at least one of IBA (indole butyric acid) and NAA (naphthalene acetic acid) at an amount between 0.1 to 10 mg/L based on the total volume of the culture medium, and the culture medium contains 10-100 g/L of sucrose based on the total volume of the culture medium.

7. The culturing method of claim 6, wherein in the step (b), the culture medium contains 2 to 8 mg/L of IBA (indole butyric acid) based on the total volume of the culture medium.

8. The culturing method of claim 1, wherein the leguminous seeds in the step (a) are from at least one of Glycine Max Merr. and Glycine gracillis; the induced site in the step (b) is at least one of hypocotyls and radicles; and the culture medium in the step (b) contains 3 to 5 mg/L of IBA (indole butyric acid) based on the total volume of the culture medium.

9. The culturing method of claim 1, wherein in the step (c), the culture medium contains 3 to 5 mg/L of IBA (indole butyric acid) and 10-100 g/L of sucrose based on the total volume of the culture medium.

10. The culturing method of claim 1, wherein in the step (c), the culture medium is 0.5-1.5 MS medium and contains 30-60 g/L of sucrose.

11. Cultured leguminous roots having increased coumestrol content, produced by the method of claim 1.

12. The cultured leguminous roots of claim 11, wherein the content of coumestrol therein is at least 0.001 wt %.

13. Cultured leguminous roots having coumestrol content of at least 0.001 wt %.
